(19) United States
(12) Patent Application Publication
(10) Pub. No.: US 2019/0315691 A1
(43) Pub. Date: Oct. 17, 2019

(54) AN IMPROVED PROCESS FOR THE PREPARATION OF PAROXETINE AND ITS INTERMEDIATE

(71) Applicant: PIRAMAL ENTERPRISES LIMITED, Mumbai (IN)

(72) Inventors: Raghavendar Rao MORTHALA, Hyderabad (IN); Arnab ROY, Hyderabad (IN); Tapas MAITY, West Bengal (IN); Baswaraj PATLOLLA, Hyderabad (IN); Murali RAJAPPA, Hyderabad (IN); Nilanjan MAJUMDER, Hyderabad (IN); Chirag PATEL, Hyderabad (IN); Dhileepkumar KRISHNAMURTHY, Mumbai (IN); Santhosh Reddy JANGARI, Hyderabad (IN); Anant JAGTAP, Hyderabad (IN); Phani Kumar MANNEM, Hyderabad (IN); Pulendarreddy ARWAL, Hyderabad (IN)

(73) Assignee: PIRAMAL ENTERPRISES LIMITED, Mumbai (IN)

(21) Appl. No.: 15/757,027

(22) PCT Filed: Sep. 2, 2016
(86) PCT No.: PCT/IB2016/055261

§ 371 Date: Mar. 2, 2018

(30) Foreign Application Priority Data
Sep. 3, 2015 (IN) 3380/MUM/2015

(51) Int. Cl. C07D 211/22; C07D 405/12

(52) U.S. Cl. CPC C07D 211/22; C07D 405/12

(57) ABSTRACT

The present invention provides an improved process for the preparation of N-protected ((3S,4R)-4-(4-fluorophenyl)piperidin-3-yl)methanol (compound (A)) and further its transformation to Paroxetine and its pharmaceutically acceptable salts. The process comprises reaction of compound (II) with amido-malonate compound (C) in the presence of a chiral catalyst and optionally a dehydrating agent to obtain compound (B); followed by reduction of (B) in the presence of a reducing agent to provide compound (A).

### FIELD OF THE INVENTION

The present invention relates to an improved process for the preparation of N-protected ((3S,4R)-4-(4-fluorophenyl)piperidin-3-yl)methanol (hereafter referred as the compound-A), which is useful as a key intermediate for the synthesis of Paroxetine. The process of the present invention further involves transformation of the said intermediate piperidine alcohol compound (the compound-A) to Paroxetine (referred to as the compound-I) and its pharmaceutically acceptable salts.

### BACKGROUND OF THE INVENTION

The following discussion of the prior art is intended to present the invention in an appropriate technical context, and allows its significance to be properly appreciated. Unless clearly indicated to the contrary, reference to any prior art in this specification should not be construed as an expressed or implied admission that such art is widely known or forms part of common general knowledge in the field.

Paroxetine hydrochloride (the compound I) is a compound indicated for the treatment of depression. The compound is also useful in the treatment of Obsessive Compulsive Disorder, Panic Disorder and Social Anxiety Disorder. It is the hydrochloride salt of a phenyl piperidine compound identified chemically as (-)-trans-4R-(4′-fluorophenyl)-3S-[(3′,4′-methylenedioxyphenoxy) methyl] piperidine hydrochloride hemihydrate represented by the following Formula-I. This drug is marketed under the trademark of PAXIL®.

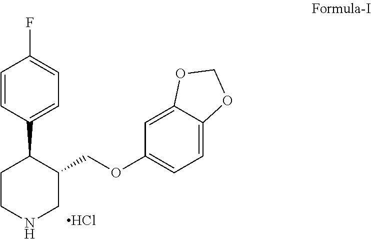 [SMILES: Fc1ccc([C@@H]2CCNC[C@H]2COc2ccc3c(c2)OCO3)cc1 ; Cl]

Paroxetine being an important drug used in treatment of depression, a number of processes for its preparation as well as for intermediate synthesis are known in the art.

U.S. Pat. No. 4,902,801 describes a process for preparation of piperidine alcohol and relates to compound (A) of the instant application comprising the reaction of amido-malonate with cinnamic acid ester and further reduction of the compound using lithium aluminium hydride or aluminium hydride, as depicted below

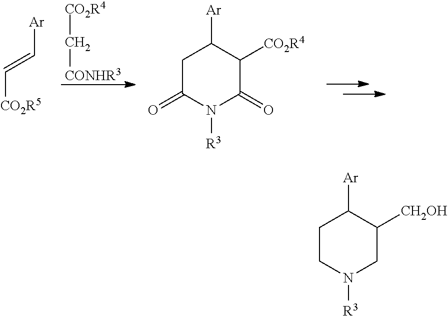

Indian patent IN 213210 disclosed preparation of (-)-trans-4-(4′-Flurophenyl)-3-hydroxymethyl-N-methyl piperidine comprising reduction of racemic dione compound using dihorane solution and further resolution of the product using di-para toluyl tartaric acid (DPTTA).

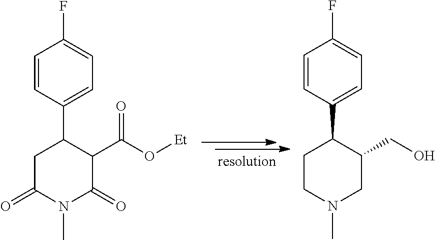 [SMILES: CCOC(=O)C1C(=O)N(C)C(=O)CC1c1ccc(F)cc1 ; CN1CC[C@@H](c2ccc(F)cc2)[C@H](CO)C1]

U.S. Pat. No. 6,444,822 describes a process for preparation of the intermediate piperidine alcohol compound (A) comprising reaction of cinnamic aldehyde with monoamide in the presence of (L)-proline rubidium salt and further reduction of the compound using lithium aluminium hydride or sodium horohydride, as depicted below

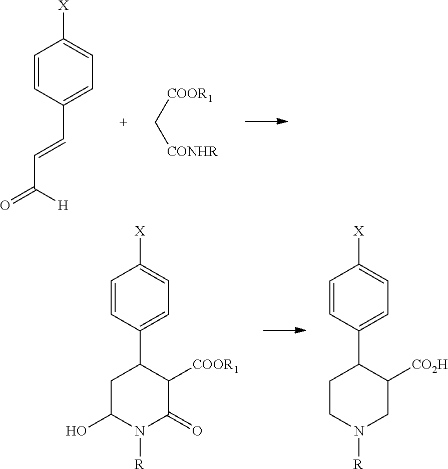

Journal article Tetrahedron 67 (2011)8942-50 and Tetrahedron letters 50 (2009) 1943-46 disclosed preparation of Paroxetine intermediates comprising reaction of 3-(4-fluorophenyl)acrylaldehyde with ethyl 3-(methylamino)-3-oxopropanoate in the presence of TMS-protected diphenylprolinol, followed by the treatment of the reaction mixture with p-toluenesulfinic acid and purification of the product by column chromatography; the product further under goes reduction using H₂-Pd/C or BH₃-THF.

Various other synthetic methods are disclosed in the U.S. Pat. No. 6,197,960, published PCT application WO-A-96/36636 and WO-A-2004/043921.

It is evident from the discussion of the processes for the preparation of Paroxetine and the intermediate N-protected ((3S,4R)-4-(4-fluorophenyl)piperidin-3-yl)methanol (the compound-A), described in the afore cited patent documents that some of the reported processes primarily involve formation of racemic compounds which need additional resolution procedure, also purification of the product using column chromatography, use of PTSA as dehydrating agent, low yield; which renders the process costlier and hence the processes are not industrially feasible.

In view of these drawbacks, there is a need to develop an industrially viable commercial process for the preparation of Paroxetine and its intermediates; which is simple, efficient and cost-effective process and provides the desired compounds in improved yield and purity.

Inventors of the present invention have developed an improved process that addresses the problem associated with the processes reported in the prior art. The process of the present invention does not involve use of any toxic and/or costly solvents and reagents. Moreover, the process does not require additional purification steps and critical workup procedure. Accordingly, the present invention provides a process for the preparation of Paroxetine and its intermediates, which is simple, efficient, cost effective, environmentally friendly and commercially scalable for large scale operations.

### SUMMARY OF THE INVENTION

In one aspect, the present invention relates to an improved process for the preparation of N-protected ((3S,4R)-4-(4-fluorophenyppiperidin-3-yl)methanol (the compound (A)), comprising reacting 3-(4-fluorophenyl)acrylaldehyde (II) with amido-malonate compound (C) in the presence of a chiral catalyst and optionally a dehydrating agent to obtain compound (B); followed by reduction of (B) in the presence of a reducing agent.

In one aspect, the present invention relates to an improved process for the preparation of N-protected ((3S,4R)-4-(4-fluorophenyl)piperidin-3-yl)methanol (the compound (A)), comprising reacting 3-(4-fluorophenyl)acrylaldehyde (II) with amido-malonate compound (C) in the presence of diphenylprolinol-TMS catalyst and molecular sieves to obtain compound (B); followed by reduction of (B) in the presence of a reducing agent.

In one aspect, the present invention relates to an improved process for the preparation of ((3S,4R)-4-(4-fluorophenyl)-1-methylpiperidin-3-yl)methanol (the compound (1A)), comprising reacting 3-(4-fluorophenyl)acrylaldehyde (II) with methyl 3-(methylamino)-3-oxopropanoate (III) in the presence of diphenylprolinol-TMS catalyst and molecular sieves to obtain compound (IV); followed by reduction of (IV) in the presence of a reducing agent.

In one aspect, the present invention relates to an improved process for the preparation of N-protected ((3S,4R)-4-(4-fluorophenyl)piperidin-3-yl)methanol (the compound (A)), comprising reacting 3-(4-fluorophenyl)acrylaldehyde (II) with amido-malonate compound (C) in the presence of diphenylprolinol-TBDMS catalyst and optionally a dehydrating agent to obtain compound (B); followed by reduction of (B) in the presence of a reducing agent.

According to another aspect of the present invention, there is provided an improved process for the preparation of ((3S,4R)-4-(4-fluorophenyl)-1-methylpiperidin-3-yl)methanol (the compound-1A), wherein the intermediate compound (IV) is obtained in a yield of about 75% and ≥99% enantiomeric purity.

In another aspect, the present invention relates to an improved process for the preparation of N-protected ((3S,4R)-4-(4-fluorophenyl)piperidin-3-yl)methanol (the compound (A)), comprising reacting 3-(4-fluorophenyl)acrylaldehyde (II) with amido-malonate compound (C) in the presence of a chiral catalyst and optionally a water removal system to obtain compound (B); followed by reduction of (B) in the presence of a reducing agent.

### DETAILED DESCRIPTION OF THE INVENTION

Accordingly, the present invention relates to an improved process for the preparation of N-protected ((3S,4R)-4-(4-fluorophenyl)piperidin-3-yl)methanol (the compound A) represented by the following formula,

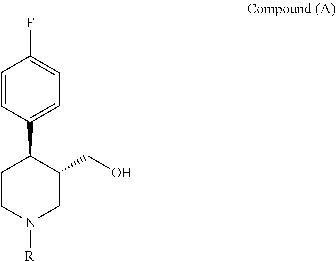

wherein R is selected from H, C₁-C₁₀ straight and/or branched alkyl, benzyl;
comprising
(i) reacting the compound (II) represented by the following formula;

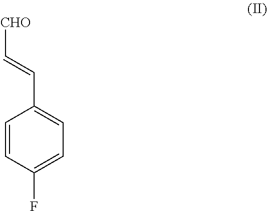 [SMILES: O=C/C=C/c1ccc(F)cc1]

with amido-malonate compound (C) represented by the following formula;

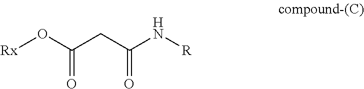

wherein Rx is H, C₁-C₁₀ straight and/or branched alkyl;
in the presence of a chiral catalyst and optionally a dehydrating agent;
(ii) reducing the compound (B) obtained from stage (i) represented by the following formula;

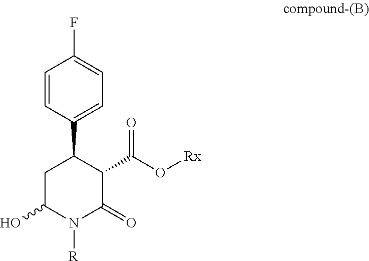

in the presence of a reducing agent.

The compound (A) obtained by afore described process is optionally, converted into Paroxetine or a pharmaceutically acceptable salt thereof.

According to another aspect, the present invention relates to an improved process for the preparation of ((3S,4R)-4-(4-fluorophenyl)-1-methylpiperidin-3-yl)methanol (the compound IA) represented by the following formula,

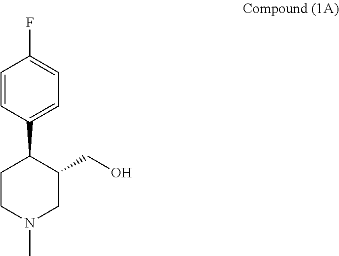 [SMILES: CN1CC[C@@H](c2ccc(F)cc2)[C@H](CO)C1]

comprising
(a) reacting the compound (II) represented by the following formula;

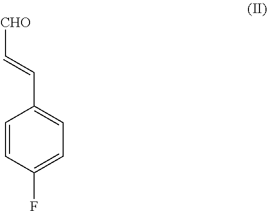 [SMILES: O=C/C=C/c1ccc(F)cc1]

with amido-malonate compound (III) represented by the following formula;

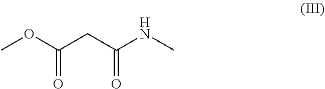

in the presence of a chiral catalyst and optionally a dehydrating agent;
(b) reducing the compound (IV) obtained from stage (a) represented by the following formula;

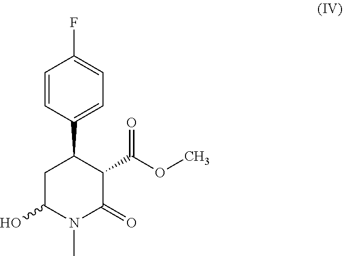 [SMILES: COC(=O)[C@@H]1C(=O)N(C)C(O)C[C@H]1c1ccc(F)cc1]

in the presence of a reducing agent to obtain the compound (1A);
(c) optionally, converting the compound (1A) into Paroxetine or a pharmaceutically acceptable salt thereof.

In the context of the present invention, the term “optionally” when used in reference to any element; including a process step e.g. conversion of a compound; it is intended to mean that the subject element is subsequently converted, or alternatively, is not converted to a further compound. Both alternatives are intended to be within the scope of the present invention.

In the context of the present invention, the term “optionally” when used in reference to any element; including a reagent e.g. dehydrating agent; it is intended to mean that the subject element is added, or alternatively, is not added during the reaction. Both alternatives are intended to he within the scope of the present invention.

In an embodiment, the chiral catalyst is selected from the group consisting of (S)-2-(diphenyl((trimethylsilyl)oxy)methyl)pyrrolidine, (S)-2-(bis(3,5-bis(trifluoromethyl) phenyl)((trimethylsilyl)oxy)methyl)pyrrolidine, (S)-2-(((tert-butyldimethylsilyl)oxy) diphenylmethyl)pyrrolidine (diphenylprolinol-TBDMS), (S)-diphenyl(pyrrolidin-2-yl)methanol, (S)-pyrrolidine-2-carboxylic acid, (S)-2-(((triethylsilyl)oxy) diphenylmethyl)pyrrolidine (diphenylprolinol-TES) and (S)-2-(((triisopropylsilyl)oxy) diphenylmethyl)pyrrolidine (diphenylprolinol-TIPS).

In an embodiment, the dehydrating agent is selected from the group consisting of molecular sieves, magnesium sulfate, calcium sulfate, sodium sulfate.

In an embodiment, the reducing agent is selected from the group consisting of hydrides such as sodium borohydride, potassium borohydride, lithium borohydride, zinc borohydride, sodium cyanoborohydride, sodium sulfurated borohydride, sodium trioxyacetal borohydride, sodium tri-alkoxy borohydride, sodium hydroxyl borohydride, sodium borohydride anilide, tetrahydrofuran borohydride, di-methyl-butyl borohydride, lithium-aluminum hydride, lithium-aluminum tri-oxymethyl hydride, sodium-aluminum-2-methoxy-ethoxy hydride, and aluminum hydride and/or mixtures thereof; borane-tetrahydrofuran (THF), borane-dimethylsulfide (DMS) and sodium horohydride/BF₃-Etherate.

In an embodiment, the reducing agent is sodium borohydride/BF₃-O(Et)₂.

In a specific embodiment, the process for the preparation of ((3S,4R)-4-(4-fluorophenyl)-1-methylpiperidin-3-yl)methanol (the compound (1A)) comprises the steps of:
(1) adding a dehydrating agent in a solvent;
(2) adding compound (II) to the stirring solution of stage (1);
(3) adding compound (III) to the stirring solution of stage (2);
(4) adding a chiral catalyst to the stirring solution of stage (3) at temperature of about 0° C.;
(5) stirring the reaction mixture of stage (4) at temperature of about 30° C.;
(6) isolating and optionally purifying the compound (IV);
(7) dissolving the compound (IV) in a solvent;
(8) adding the solution of stage (7) to the stirring solution of reducing agent in a solvent at temperature of about 10° C.; and
(9) isolating the desired product.

The process of the present invention as per the specific embodiment described above is illustrated in the following Scheme (I),

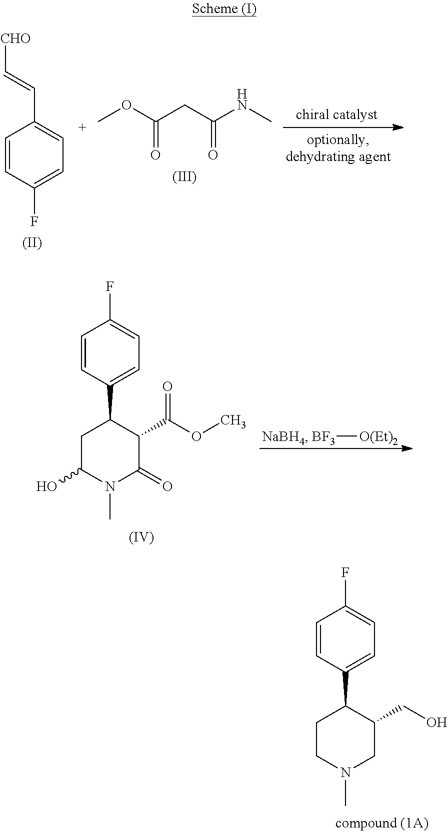

The process as described above further comprises optionally converting the pure compound (1A) into Paroxetine or a pharmaceutically acceptable salt thereof.

The solvent used in the step (1), step (7) and step (8) of the above process (as depicted in the Scheme (I)) is selected from the halogenated solvent such as dichloromethane, 4-bromotoluene, diiodomethane, carbon tetrachloride, chlorobenzene and chloroform; alcoholic solvent such as methanol, ethanol, trifluoro ethanol, isopropanol, t-amyl alcohol, t-butyl alcohol and hexanol; ketones such as Acetone; an ether solvent such as tetrahydrofuran, cyclopentyl methyl ether, 2-methyltetrahydrofuran, diethyl ether and 1,4-dioxane; an ester solvent such as ethyl acetate, isopropyl acetate, propyl acetate and butyl acetate; an aprotic solvent such as acetonitrile; an aromatic solvent such as toluene, xylene and benzene; water and/or a mixture thereof.

The dehydrating agent used in the step (1) of the above process (as depicted in the Scheme (I)) is selected from molecular sieves, magnesium sulfate, calcium sulfate, sodium sulfate.

The chiral catalyst used in the step (4) of the above process (as depicted in the Scheme (I)) is selected from the group consisting of (S)-2-(diphenyl((trimethylsilyl)oxy)methyl)pyrrolidine, (S)-2-(bis(3,5-bis(trifluoromethyl) phenyl)((trimethylsilyl)oxy)methyl)pyrrolidine, (S)-2-(((tert-butyldimethylsilyl)oxy) diphenylmethyl)pyrrolidine (diphenylprolinol-TBDMS), (S)-diphenyl(pyrrolidin-2-yl)methanol, (S)-pyrrolidine-2-carboxylic acid, (S)-2-(((triethylsilyl)oxy) diphenylmethyl)pyrrolidine (diphenylprolinol-TES) and (S)-2-(((triisopropylsilyl)oxy) di phenylmethyl)pyrrolidine (diphenylprolinol-TIPS).

The term ‘temperature of about 0° C.’ referred to in the step (4) of the above process (as depicted in the Scheme (I)) can range from −10° C. to +10° C. More preferably, the temperature ranges from −5° C. to +5° C.

The term ‘temperature of about 30° C.’ referred to in the step (5) of the above process (as depicted in the Scheme (I)) can range from 20° C. to 40° C. More preferably, the temperature ranges from 25° C. to 35° C.

The term ‘isolating’ referred to in the step (6) and step (9) corresponds to the steps involving filtration, addition of water, extraction, precipitation, separation of solvents, evaporation of solvent, crystallization, filtration, washing and drying.

The reducing agent used in the step (8) of the above process (as depicted in the Scheme-I) is selected from hydrides such as sodium borohydride, potassium borohydride, lithium borohydride, zinc borohydride, sodium cyanoborohydride, sodium sulfurated horohydride, sodium trioxyacetal borohydride, sodium tri-alkoxy horohydride, sodium hydroxyl borohydride, sodium horohydride anilide, tetrahydrofuran horohydride, di-methyl-butyl horohydride, lithium-aluminum hydride, lithium-aluminum tri-oxymethyl hydride, sodium-aluminum-2-methoxy-ethoxy hydride, and aluminum hydride and/or mixtures thereof; horane-tetrahydrofuran (THF), borane-dimethylsulfide (DMS) and sodium horohydride/BF₃-Etherate.

The term ‘temperature of about 10° C.’ referred to in the step (8) of the above process (as depicted in the Scheme I) can range from −5° C. to +15° C. More preferably, the temperature ranges from 0° C. to 10° C.

The process of the present invention as illustrated in the above Scheme (I) comprises addition of 3-(4-fluorophenyl) acrylaldehyde (II) in trifluoro ethanol containing molecular sieves) (4A°). To the stirring solution was added methyl 3-(methylamino)-3-oxopropanoate (III) and (S)-2-(diphenyl((trimethylsilyl)oxy)methyl)pyrrolidine. The solvent was evaporated and product was extracted with methylene chloride. The organic layer was washed with sodium carbonate solution and the separated organic layer was evaporated to provide compound (IV) with a crude yield of about 50% and after crystallization using MTBE solvent pure (IV) was obtained with a yield of about 38% with an improved ee (enantiomeric excess). The solution of compound (IV) in THF was added to the mixture of sodium borohydride/BF₃-etherate in THF and the desired product ((3S,4R)-4-(4-fluorophenyl)-1-methylpiperidin-3-yl)methanol (the compound (1A)) was obtained with a yield of about 78%, ee of about 97% and ≥98.5% HPLC purity.

The enantiomerically pure compound (1A) as obtained by the process of the present invention can be further converted to Paroxetine or its salts. For instance such a conversion of compound (1A) to Paroxetine can be made by following the process disclosed in U.S. Pat. No. 3,912,743 and U.S. Pat. No. 4,007,196 (hereafter US '196). The process disclosed in US '196 involves reaction of the compound-1A with 1 3-benzodioxole chloride.

Advantageously, the process of the present invention provides intermediate compound (IV) with significant improvements in the yield of about 50-65% and improved ee over the processes reported in the prior art. Also the process provides product compound (1A) with an improved yield of about 70-80%, ee of about 97% and ≥98.5% HPLC purity. Eventually, the process of the instant invention effectively contributes to the reduction of the overall cost of the process. Hence, the process of the present invention is simpler and it overcomes the drawbacks of the known methods.

Accordingly in yet another aspect, the present invention relates to an improved process for the preparation of ((3S,4R)-4-(4-fluorophenyl)-1-methylpiperidin-3-yl)methanol (the compound 1A), comprising reacting 3-(4-fluorophenyl)acrylaldehyde (II) with methyl 3-(methylamino)-3-oxopropanoate (III) in the presence of a chiral catalyst and molecular sieves to obtain compound (IV); followed by reduction of compound (IV) in the presence of a reducing agent.

In a general aspect of present invention, the dehydrating agent is molecular sieves.

The dehydrating agent used in the reaction of the instant invention is a drying agent which helps to remove water from reaction. Molecular sieves are typically zeolite compounds that strongly adsorb water and have carefully controlled pore sizes. While both the solvent and the water will adsorb strongly to the molecular sieve surfaces, the large surface area within the pores is only accessible to the smaller water molecules, so they are effectively removed from the solvent. Water is able to occupy the large surface area inside the pores and thus get removed.

In yet another general aspect, the present invention relates to an improved process for the preparation of N-protected ((3S,4R)-4-(4-fluorophenyl)piperidin-3-yl)methanol (the compound-A), comprising reacting 3-(4-fluorophenyl)acrylaldehyde (II) with amido-malonate compound (C) in the presence of a chiral catalyst and a water removal system to obtain compound (B); followed by reduction of compound (B) in the presence of a reducing agent.

In a general aspect, the water removal system refers to the azeotropic removal of water from the reaction mixture. The azeotropic separation of water is achieved by the use of Dean-Stark apparatus.

In yet another aspect, the present invention relates to an improved process for the preparation of N-protected ((3S,4R)-4-(4-fluorophenyl)piperidin-3-yl)methanol (the compound (A)), comprising reacting 3-(4-fluorophenyl)acrylaldehyde (II) with amido-malonate compound (C) in the presence of diphenylprolinol-TBDMS catalyst and optionally a dehydrating agent to obtain compound (B); followed by reduction of compound (B) in the presence of a reducing agent.

In one embodiment, the present invention is directed to a process for the preparation of ((3S,4R)-4-(4-fluorophenyl)-1-methylpiperidin-3-yl)methanol (the compound 1A), wherein 3-(4-fluorophenyl)acrylaldehyde (II) reacts with methyl 3-(methylamino)-3-oxopropanoate (III) in the presence of diphenylprolinol-TBDMS catalyst.

In one embodiment, the present invention is directed to a process for the preparation of ((3S,4R)-4-(4-fluorophenyl)-1-methylpiperidin-3-yl)methanol (the compound 1A), wherein 3-(4-fluorophenyl)acrylaldehyde (II) reacts with methyl 3-(methylamino)-3-oxopropanoate (III) in the presence of diphenylprolinol-TBDMS catalyst and in absence of a dehydrating agent.

The process of the present invention as illustrated in the above Scheme (II) comprises addition of 3-(4-fluorophenyl) acrylaldehyde (II) to the stirring solution of methyl 3-(methylamino)-3-oxopropanoate (III) in ethyl acetate, (S)-2-(((tert-butyldimethylsilyl)oxy) diphenylmethyl) pyrrolidine (diphenylprolinol-TBDMS) and (potassium acetate) KOAc. The product was extracted with ethyl acetate. The organic layer was washed with sodium carbonate solution and the separated organic layer was evaporated to provide pure compound (IV) with a yield of about 57% with an improved ee of ≥98%. The solution of compound (IV) in THF was added to the mixture of sodium borohydride/BF₃-etherate in THF and the desired pure product ((3S,4R)-4-(4-fluorophenyl)-1-methylpiperidin-3-yl)methanol (the compound (1A)) was obtained with a yield of about 70-80%.

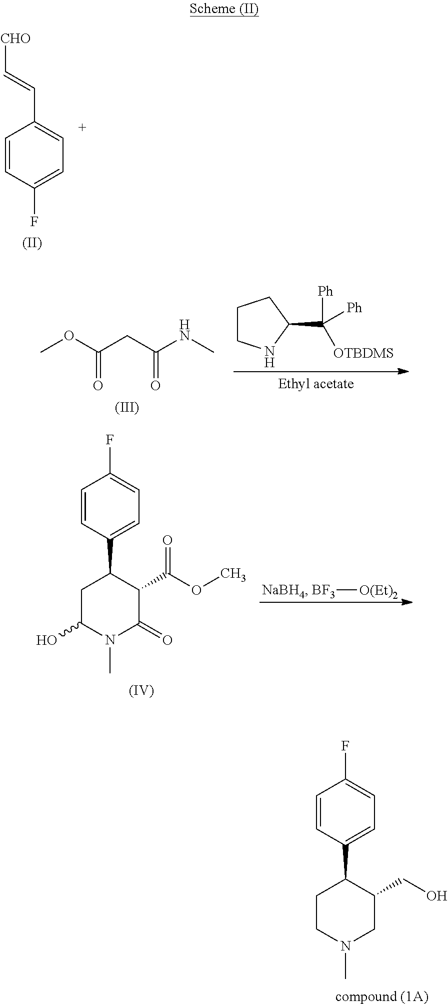

It is now evident that the diphenylprolinol-TBDMS is a stable and bulkier catalyst compared to TMS catalyst as used in the prior art processes. The use of diphenylprolinol-TBDMS improved the product yield to about 50-65% and increased enantioselectivity from about 75% to 90% (crude ee) as compared to the prior art procedure. It is also observed that the rate of hydrolysis of the catalyst is much lower or negligible compared to the prior processes.

It is also evident that the bulkier catalyst provides higher enantioselectivity such as for diphenylprolinol-TES (enantioselectivity 80-90%) and for diphenylprolinol-TIPS (enantioselectivity 90-94%).

The invention is further illustrated by the following examples which are provided to be exemplary of the invention, and do not limit the scope of the invention. While the present invention has been described in terms of its specific embodiments, certain modifications and equivalents will be apparent to those skilled in the art and are intended to be included within the scope of the present invention.

### EXAMPLES

### Example-1: Preparation of (3S,4R)-methyl 4-(4-fluorophenyl)-6-hydroxy-1-methyl-2-oxopiperidine-3-carboxylate (IV)

Charged 2,2,2-trifluoro ethanol (400 mL) and 4A° molecular sieves (25 g) in a flask followed by the addition of 3-(4-fluorophenyl)acrylaldehyde (II) (50 g) and methyl 3-(methylamino)-3-oxopropanoate (III) (52.7 g). The reaction mixture was cooled to 0° C. To the cooled reaction mixture was added (S)-2-(diphenyl((trimethylsilyl)oxy)methyl)pyrrolidine (21.68 g) and potassium acetate (39.22 g), stirred for about 19 h at temperature of about 30° C. Solvent was evaporated and product was extracted with methylene chloride (500 mL). The organic layer was washed with sodium carbonate solution and the separated organic layer was evaporated to provide crude compound (IV) with a yield of 50%. The crude product was crystallized using MTBE solvent providing solid pure compound (IV) with a yield of 38%.

### Example-2: Preparation of ((3S,4R)-4-(4-fluorophenyl)-1-methylpiperidin-3-yl)methanol (the compound (1A))

Charged THF (100 mL) and Sodium Borohydride (28.11 g) in a flask; followed by the addition of BF₃-Etherate (98.9 g) at a temperature of about 10-15° C. To the said reaction mixture was slowly added solution of compound (IV) (50 g) in THF (230 mL). The reaction mixture was stirred for 3 h at a temperature of about 25-30° C. The reaction mixture was treated with 200 mL of 3N HCl solution and was added 150 mL of toluene. The reaction mixture was treated with NaOH solution and the separated organic layer was evaporated to obtain desired product ((3S,4R)-4-(4-fluorophenyl)-1-methylpiperidin-3-yl)methanol (the compound 1A) with a yield of 78%, ee of 97% and ≥98.5% HPLC purity.

### Example-3: Preparation of (3S,4R)-methyl 4-(4-fluorophenyl)-6-hydroxy-1-methyl-2-oxopiperidine-3-carboxylate (IV)

Charged ethyl acetate (45 mL) in a flask followed by the addition of methyl 3-(methylamino)-3-oxopropanoate (III) (10.5 g, 40 mmol) at room temperature. To the stirring solution was added 4-fluoroinnamaldehyde (II) (5 g, 33 mmol), (S)-2-(((tert-butyldimethylsilyl)oxy) diphenylmethyl) pyrrolidine (diphenylprolinol-TBDMS) catalyst (0.98 g, 2.7 mmol) and potassium acetate (KOAc) (4.58 g, 47 mmol), and stirred for about 12-16 h at 45-50° C. The reaction mixture was cooled to about 25° C. and washed with 5% aq. NaHCO₃ solution (500 mL). The separated organic layer was washed with 5% brine solution (500 mL) and evaporated under reduced pressure to obtain a crude product. The crude product was purified with toluene (600 mL) to obtain pure solid product (5.3 g, 57% yield) and >99% enantiomeric purity.

### Example-4: Preparation of ((3S,4R)-4-(4-fluorophenyl)-1-methylpiperidin-3-yl)methanol (the compound (1A))

Charged THF (30 mL) and (3S,4R)-methyl 4-(4-fluorophenyl)-6-hydroxy-1-methyl-2-oxopiperidine-3-carboxylate (IV) (5 g, 17.8 mmol) in a flask, followed by the addition of sodium borohydride (2.86 g, 74 mmol) and BF₃-Etherate solution below −10° C. under nitrogen atmosphere. The reaction mixture was gradually warmed to 25-30° C. and continued stirring for 5-6 h. The reaction mixture was quenched with 3N HCl solution and the solvent was evaporated under reduced pressure. The reaction mass was diluted with demineralized (DM) water (15 mL), followed by addition of toluene (25 mL). The mixture was cooled to 5° C. and the pH was adjusted to 12-14 using Lye solution at below 20° C. The mixture was filtered and the separated organic layer was evaporated completely under vacuum to obtain crude compound. The product was further purified by treatment with toluene (5 mL) and n-heptane (5 mL) to obtain the pure product (3.17 g, 80% yield), % ee: 97%, % HPLC purity: ≥98.5%.

1. A process for the preparation of N-protected ((3S,4R)-4-(4-fluorophenyl)piperidin-3-yl)methanol (A) of the following formula, 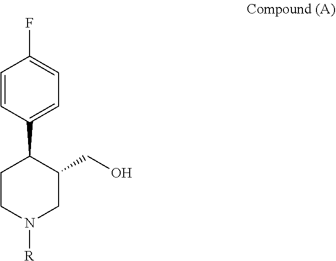
wherein R is selected from H, C₁-C₁₀ straight and/or branched alkyl, benzyl;
comprising;
(i) reacting the compound (II) of the following formula; 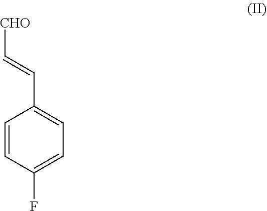 [SMILES: O=C/C=C/c1ccc(F)cc1]
with amido-malonate compound (C) represented by the following formula; 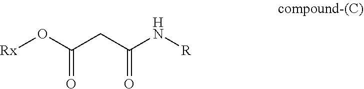
wherein Rx is H, C₁-C10 straight and/or branched alkyl;
in the presence of a chiral catalyst and optionally a dehydrating agent;
(ii) reducing the compound (B) of the following formula obtained from stage (i); 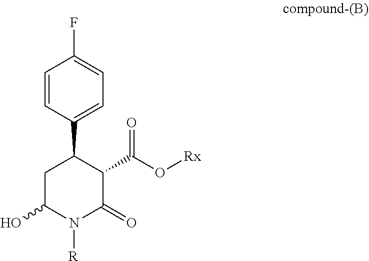
in the presence of a reducing agent.

2. The process according to the claim I, wherein the chiral catalyst is selected from the group consisting of (S)-2-(diphenyl((trimethylsilyl)oxy)methyl)pyrrolidine, (S)-2-(bis(3,5-bis(trifluoromethyl) phenyl)((trimethylsilyl)oxy)methyl)pyrrolidine, (S)-2-(((tert-butyldimethyl)silyl)oxy) diphenylmethyl)pyrrolidine (diphenylprolinol-TBDMS), (S)-diphenyl(pyrroidin-2-yl)ethanol, (S)-pyrrolidine-2-carboxylicacid, (S)-2-(((triethylsilyl)oxy) diphenylmethyl)pyrrolidine (diphenylprolinol-TES) and (S)-2-(((tri isopropylsilyl)oxy) diphenylmethyl)pyrrolidine (diphenylprolinol-TIPS).

3. The process according to the claim 1, wherein the dehydrating agent is selected from the group consisting of molecular sieves, magnesium sulfate, calcium sulfate or sodium sulfate.

4. The process according to the claim 1, wherein the reducing agent is selected from the group consisting of hydrides selected from sodium borohydride, potassium borohydride, lithium borohydride, zinc borohydride, sodium cyanoborohydride, sodium sulfurated borohydride, sodium trioxyacetal borohydride, sodium tri-alkoxy borohydride, sodium hydroxyl borohydride, sodium borohydride anilide, tetrahydrofuran borohydride, di-methyl-butyl borohydride, lithium-aluminum hydride, lithium-aluminum tri-oxymethyl hydride, sodium-aluminum-2-methoxy-ethoxy hydride, and aluminum hydride and/or mixtures thereof; borane-tetrahydrofuran (THF), borane-dimethylsulfide (DMS) and sodium borohydride/BF₃-Etherate.

5. A process for the preparation of ((3S,4R)-4-(4-fluorophenyl)-1-methylpiperidin-3-yl)methanol (1A) of the following formula, 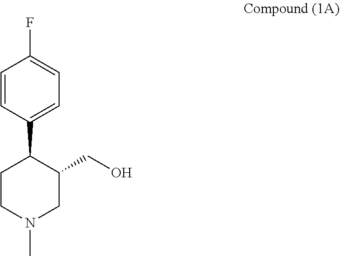 [SMILES: CN1CC[C@@H](c2ccc(F)cc2)[C@H](CO)C1]
comprising
(a) reacting the compound (II) of the following formula; 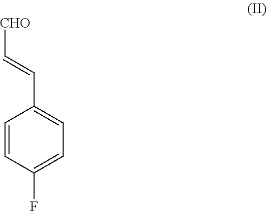 [SMILES: O=C/C=C/c1ccc(F)cc1]
with methyl 3-(methylamino)-3-oxopropanoate (III) represented by the following formula; 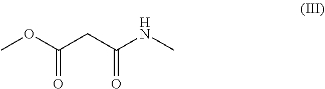
in the presence of a (S)-2-(((tert-butyldimethylsilyl)oxy) diphenylmethyl)pyrrolidine (diphenylprolinol-TBDMS) of the following formula; 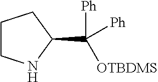 [SMILES: CC(C)(C)[Si](C)(C)OC(c1ccccc1)(c1ccccc1)[C@@H]1CCCN1]
(b) reducing the compound (IV) of the following formula obtained from stage (a); 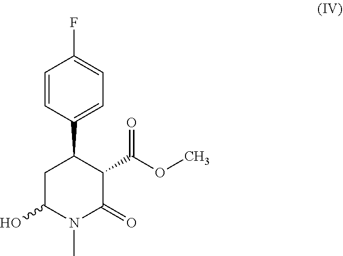 [SMILES: COC(=O)[C@@H]1C(=O)N(C)C(O)C[C@H]1c1ccc(F)cc1]
in the presence of sodium borohydride/BF₃-O(Et)₂.

6. A process for the preparation of ((3S,4R)-4-(4-fluorophenyl)-1-methylpiperidin-3-yl)methanol (1A) of the following formula, 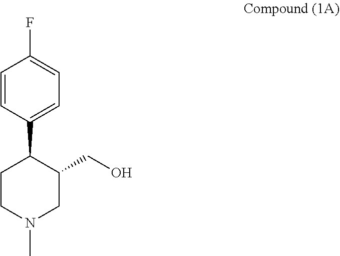 [SMILES: CN1CC[C@@H](c2ccc(F)cc2)[C@H](CO)C1]
comprising
(ai) reacting the compound (II) of the following formula; 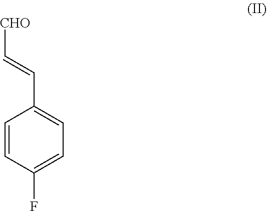 [SMILES: O=C/C=C/c1ccc(F)cc1]
with methyl 3-(methylamino)-3-oxopropanoate (III) represented by the following formula; 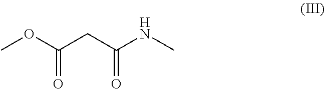
in the presence of (S)-2-(diphenyl((trimethylsilyl)oxy)methyl)pyrrolidine and a dehydrating agent;
(bi) reducing the compound (IV) of the following formula obtained from stage (ai); 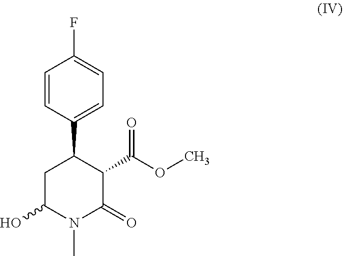 [SMILES: COC(=O)[C@@H]1C(=O)N(C)C(O)C[C@H]1c1ccc(F)cc1]
in the presence of sodium borohydride/BF₃-O(Et)₂.

7. The process according to claim 1, wherein the compound (A) or the compound (I A) is further converted to Paroxetine or its pharmaceutically acceptable salt.

8. The process according to claim 5, wherein the compound (A) or the compound (1A) is further converted to Paroxetine or its pharmaceutically acceptable salt.

9. The process according to claim 6, wherein the compound (A) or the compound (1A) is further converted to Paroxetine or its pharmaceutically acceptable salt.
